(19) United States
(12) Patent Application Publication
(10) Pub. No.: US 2023/0220003 A1
(43) Pub. Date: Jul. 13, 2023

(54) PURIFICATION METHOD FOR IMIDAZOLE DIPEPTIDES

(71) Applicant: TOKAI BUSSAN CO., LTD., Tokyo (JP)

(72) Inventors: Akira YONEYAMA, Tokyo (JP); Chiaki SANO, Tokyo (JP); Hiroki NAKANISHI, Tokyo (JP); Yoshio KAWAI, Tokyo (JP); Yohsuke KOYAMA, Tokyo (JP)

(73) Assignee: TOKAI BUSSAN CO., LTD., Tokyo (JP)

(21) Appl. No.: 17/997,370

(22) PCT Filed: Apr. 28, 2021
(86) PCT No.: PCT/JP2021/016910

§ 371 Date: Oct. 28, 2022

(30) Foreign Application Priority Data
Apr. 30, 2020 (JP) 2020-080065

(51) Int. Cl. C07K 1/20; C07K 5/06; C07D 233/64; B01D 15/32

(52) U.S. Cl. CPC C07K 1/20; C07K 5/06; C07D 233/64; B01D 15/327

(57) ABSTRACT

The objective of the present invention is to provide a method of producing each high purity imidazole dipeptide in large quantities on an industrial scale, regardless of the type of animal extract employed. The objective is achieved by a method of producing a purified imidazole dipeptide composition, including the step (1) of subjecting an animal extract treatment solution containing at least two types of imidazole dipeptides to adsorption treatment carried out by bringing the solution into contact with a hydrophobic adsorption resin to adsorb the imidazole dipeptides onto the hydrophobic adsorption resin; and the step (2) of subjecting the hydrophobic adsorption resin adsorbing the imidazole dipeptides to elution treatment using an aqueous solution to mutually separate and collect the imidazole dipeptides to purify an imidazole dipeptide.

### TECHNICAL FIELD

The present invention relates to a method for purifying imidazole dipeptides.

### BACKGROUND ART

Imidazole dipeptides are dipeptides formed by binding of histidine or a histidine derivative having an imidazole group with an amino acid. Specific examples of imidazole dipeptides include anserine (β-alanyl-1-methylhistidine), carnosine (β-alanyl histidine), balenine (β-alanyl-3-methylhistidine) and homocarnosine (γ-aminobutyryl-L-histidine). Such imidazole dipeptides are known to have physiological effects such as anti-fatigue effect, antioxidant effect, hypoglycemic effect and cognition function improvement effect, and have attracted attention as a functional ingredient.

Known methods of producing the imidazole dipeptides include methods of chemically, enzymatically, or microbiologically synthesizing imidazole dipeptides in the use of L-histidine, 3-methyl-L-histidine and the like as starting materials. For example, Patent Document 1 as listed below (the entire disclosure of which is incorporated by reference herein) discloses a method of producing imidazole dipeptides using a microorganism having the activity of synthesizing the imidazole dipeptides. However, since the method according to Patent Document 1 employs 3-methyl-L-histidine and 1-methyl-L-histidine as the starting materials, it is still challenging to produce imidazole dipeptides in large quantity on an industrial scale from the viewpoint of stably supplying the starting materials.

On the other hand, the methods of producing imidazole dipeptides in large quantities on an industrial scale include methods of obtaining imidazole dipeptides from extracts of animals such as fish including tuna, bonito and salmon, mammals including cattle, pig and whale, and birds including chicken.

The methods of producing imidazole dipeptides from an animal extract include methods with the use of ion exchange treatment. For example, Patent Document 2 as listed below (the entire disclosure of which is incorporated by reference herein) discloses a method including the step of adsorbing imidazole dipeptides by passing a demineralized solution obtained by demineralizing a fish-and-shellfish extract through an H type weakly acidic cation exchange resin, and the step of washing the resin with water followed by eluting the imidazole dipeptides with hydrochloric acid and/or brine.

Patent Document 3 as listed below (the entire disclosure of which is incorporated by reference herein) discloses a method of adsorbing imidazole dipeptides by bringing an animal extract into contact with a strongly acidic cation exchange resin which was equilibrated to H type in advance using a buffer solution adjusted to the same ranges of electrical conductivity (10±2 mS/cm) and pH (5.0±0.5) as those of the animal extract, washing the resin with the buffer solution and pure water, and then eluting the imidazole dipeptides by passing an alkaline solution in the range between pH 8 and pH 12 through the resin or mixing them.

### CITATION LIST

### Patent Documents

[Patent Document 1] JP 2020-22433 A
[Patent Document 2] JP 4612549 B
[Patent Document 3] JP 5142126 B

### SUMMARY OF THE INVENTION

### Problems to be Solved by the Invention

In general, the animal extract contains two or more types of imidazole dipeptides, depending on the type and part of the animal. However, since the imidazole dipeptides such as anserine, carnosine, balenine and homocarnosine have the similar electrical property to each other, the method using ion exchange treatment described in Patent Document 2 or 3 cannot individually separate two or more types of imidazole dipeptides, and thus collects the imidazole dipeptides as they are in the original ratio contained in the animal extract. In other words, the problem is that the method using ion exchange treatment described in Patent Document 2 or 3 cannot mutually separate two or more types of imidazole dipeptides.

The molecular weight of carnosine is 226, and the molecular weights of homocarnosine, anserine and balenine are 240. Since the molecular weights of the imidazole dipeptides are very similar to each other, the problem is that membrane fractionation due to molecular weight using reverse osmosis or nanofiltration (NF) membranes cannot mutually separate two or more types of imidazole dipeptides.

For example, the imidazole dipeptides obtained by applying ion exchange treatment using a chicken derived extract would become a mixture containing anserine and carnosine at a ratio of 2:1 to 3:1 by weight. If a whale derived extract is employed, the resulting imidazole dipeptides would become a mixture containing balenine and carnosine at a ratio of 4:1 to 5:1 by weight. Few methods for mutually separating each imidazole dipeptide from the mixtures are known so far.

On the other hand, since most of the imidazole dipeptides contained in salmon are anserine, a highly pure anserine can be obtained by subjecting a salmon derived extract to a method carrying out ion exchange treatment. However, the problems are that the number of animal species with a single imidazole dipeptide is limited, and salmon is dependent on catch quantity.

Therefore, the problems still remain that there are few methods so far to produce each high purity imidazole dipeptide in large quantities on an industrial scale, regardless of the type of the animal extract employed.

In view of the above circumstances, it is an objective of the present invention to provide a method of producing each high purity imidazole dipeptide in large quantities on an industrial scale, regardless of the type of the animal extract employed.

### Means for Solving the Problems

In order to solve the problems, the present inventors conducted extensive research and focused on the fact that anserine and balenine have a structure in which one methyl group is attached to an imidazole ring, differing from carnosine. Then, depending on such a structural difference, the present inventors repeated trial and error on mutual separation based on the differences in hydrophobicity between the molecular species.

Surprisingly, the present inventors found that imidazole dipeptides could be mutually separated by subjecting an animal extract treatment solution containing two or more types of imidazole dipeptides to adsorption treatment using a hydrophobic adsorption resin and elution treatment using a specific eluent.

Based on such findings, the present inventors finally succeeded in creating a method of producing a purified imidazole dipeptide composition containing an individual high purity imidazole dipeptide from an animal extract treatment solution containing at least two types of imidazole dipeptides. As such, the present invention has been completed on the basis of the findings and successful examples.

According to the present invention, there is provided methods according to the following aspects [1] to [9]:

[1] A method of producing a purified imidazole dipeptide composition, including the steps of:

(1) subjecting an animal extract treatment solution containing at least two types of imidazole dipeptides to adsorption treatment carried out by bringing the solution into contact with a hydrophobic adsorption resin to adsorb the imidazole dipeptides onto the hydrophobic adsorption resin; and

(2) subjecting the hydrophobic adsorption resin adsorbing the imidazole dipeptides to elution treatment using an aqueous solution to mutually separate and collect imidazole dipeptides to purify an imidazole dipeptide.

[2] The method according to [1], wherein the at least two types of imidazole dipeptides contain carnosine and an imidazole dipeptide different from carnosine.

[3] The method according to [1], wherein the at least two types of imidazole dipeptides contain carnosine, and anserine or balenine.

[4] The method according any one of [1] to [3], wherein the hydrophobic adsorption resin is an aromatic hydrophobic adsorption resin.

[5] The method according to any one of [1] to [4], wherein the aqueous solution is at least one aqueous solution selected from the group consisting of water, a dilute alkaline aqueous solution and a dilute organic solvent aqueous solution.

[6] The method according to [5], wherein the dilute alkaline aqueous solution is 0.001 M to 0.008 M sodium hydroxide aqueous solution, and the dilute organic solvent aqueous solution is 0.1 wt % to 0.8 wt % ethanol aqueous solution.

[7] The method according to any one of [1] to [6], wherein the animal extract treatment solution is obtained by subjecting an animal extract to ion adsorption treatment using a strongly acidic cation exchange resin followed by elution treatment using an alkaline aqueous solution.

[8] The method according to [7], wherein the animal extract is an animal extract subjected to demineralization treatment.

[9] The method according to [7] or [8], wherein the animal extract is derived from meat of at least one animal selected from the group consisting of chicken, whale, cattle, pig, salmon, bonito and tuna.

### Advantageous Effects of the Invention

Since the method according to one embodiment of the present invention employs adsorption treatment using a hydrophobic adsorption resin and elution treatment using a specific eluent, a purified imidazole dipeptide composition containing each high purity imidazole dipeptide can be obtained without using complicated facilities, equipments or operations through the method. Therefore, the method according to one embodiment of the present invention is a simple and economical method so that the method can be carried out on an industrial scale.

By using the purified imidazole dipeptide composition obtained by the method according to one embodiment of the present invention, it is expected to achieve physiological effects inherent in any one of imidazole dipeptides such as anserine, balenine and carnosine.

### BRIEF DESCRIPTION OF THE DRAWINGS

FIG. 1 is the graph illustrating the example separation obtained by mutually separating the mixture of purified anserine and carnosine compositions, as described in Examples below.

FIG. 2 is the graph illustrating the example separation obtained by mutually separating anserine and carnosine using each of eluents, as described in Examples below.

FIG. 3 is the graph illustrating the example separation obtained by mutually separating anserine and carnosine using DIAION HP20 as a synthetic adsorption resin, as described in Examples below.

FIG. 4 is the graph illustrating the HPLC chromatograms of the chicken extract, the ion exchange treatment solution and the ion exchange treatment+NF membrane treatment solution, as described in the Examples below.

FIG. 5 is the graph illustrating the example separation obtained by mutually separating anserine and carnosine, using chicken as a raw material, as described in Examples below.

FIG. 6 is the graph illustrating the HPLC chromatograms of the crude anserine purified solution, the first half fraction collected, and the second half fraction collected, as described in the Examples below.

FIG. 7 is the graph illustrating the HPLC chromatograms of the whale extract, the ion exchange treatment solution and the ion exchange treatment+NF membrane treatment solution, as described in the Examples below.

FIG. 8 is the graph illustrating the example separation obtained by mutually separating anserine and carnosine using whale meat as a raw material, as described in Examples below.

FIG. 9 is the graph illustrating the HPLC chromatograms of the crude balenine purified solution, the first half fraction collected, and the second half fraction collected, as described in the Examples below.

FIG. 10 is the graph illustrating the example separation obtained by mutually separating carnosine, anserine and balenine using pork as a raw material, as described in Examples below.

FIG. 11 is the graph illustrating the HPLC chromatograms of the crude carnosine purified solution, the first half fraction collected, and the second half fraction collected, as described in the Examples below.

### MODES FOR CARRYING OUT THE INVENTION

While each method that forms one embodiment of the present invention will now be described in detail, the present invention is not limited only by the matters of this section, and may take various forms to the extent that its objective can be achieved.

Unless otherwise specified, each term in this specification is used in the sense normally used by those skilled in the technical fields such as foods, pharmaceuticals and cosmetics and should not be construed as having an unduly restrictive meaning. Also, any speculations and theories herein are made on the basis of the knowledge and experiences of the present inventors and as such, the present invention is not bound by any such speculations and theories.

The term “RV” means a multiple number of flow rate of solvent relative to an amount of resin. For example, if the two times amount of animal extract relative to the amount of resin is passed through the resin, RV makes 2.

The term “SV” is Space Velocity, which means a ratio per hour of a liquid amount (volume) flowed to a resin amount (volume). For example, if 5 m³ of liquid is passed through 1 m³ of resin for an hour, SV makes 5.

The term “and/or” as used herein means either any one of, any combination of two or more of, or any combination of all of listed related items.

The wording “to” for indicating a range of values is intended to include values preceding and following the wording; for example, “0 wt % to 100 wt %” means a range from 0 wt % or more and 100 wt % or less. As used herein, the term “wt %” is synonymous with “% by mass” or “% (w/w)”.

The terms “include,” “comprise,” and “contain” mean that an element(s) other than an element(s) as explicitly indicated can be added as inclusions, which are, for example, synonymous with “at least include,” but encompasses the meaning of “consist of” and “substantially consist of”. In other words, the terms may mean, for example, to include an element(s) as explicitly indicated as well as any one element or any two or more elements, to consist of an element(s) as explicitly indicated, or to substantially consist of an element(s) as explicitly indicated. Such elements include limitations such as components, steps, conditions, and parameters.

The number of digits of an integer equals to its significant figure. For example, 1 has one significant figure and 10 has two significant figures. For a decimal number, the number of digits after a decimal point equals to its significant figure. For example, 0.1 has one significant figure and 0.10 has two significant figures.

### Summary of Method According to One Embodiment of the Present Invention

The method according to one embodiment of the present invention relates to a method of producing a purified imidazole dipeptide composition containing a single high purity imidazole dipeptide from an animal extract treatment solution which is obtained by subjecting an animal extract to ion exchange treatment and the like and contains at least two types of imidazole dipeptides.

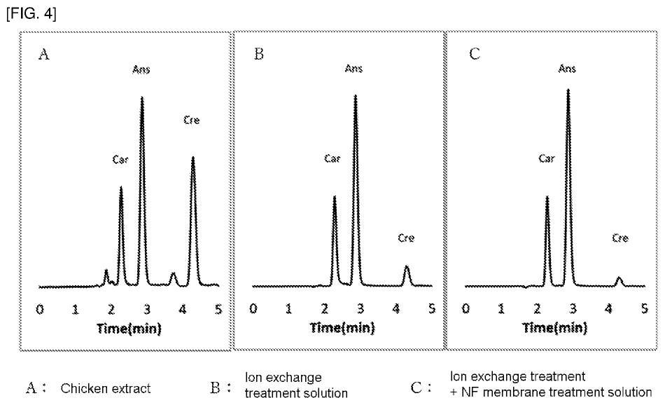

For example, as shown in FIG. 4, when a chicken extract is subjected to adsorption treatment with a strongly acidic cation exchange resin, the adsorbed imidazole dipeptides are eluted with caustic soda, and then the resulting eluate is subjected to demineralization treatment with an NF membrane, and a highly pure imidazole dipeptide composition with creatinine removed is obtained. However, the constituent ratio of imidazole dipeptides (the content ratio of anserine and carnosine) remains almost the same as in the chicken extract, the ion exchange treatment solution or the ion exchange treatment+NF membrane treatment solution.

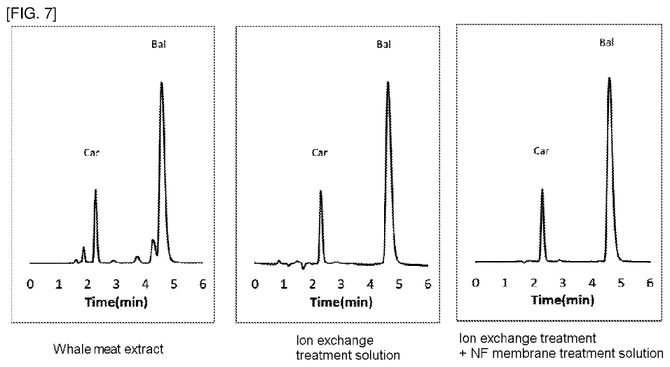

In the similar manner, as shown in FIG. 7, when a whale meat extract is used, the content ratio of balenine and carnosine in the resulting imidazole dipeptide composition is almost unchanged through each treatment.

Thus, if an animal extract is simply subjected to ion exchange treatment, the constituent ratio of imidazole dipeptides such as anserine, balenine and carnosine in the resulting treatment product would be determined due to the type of animal in the animal extract used.

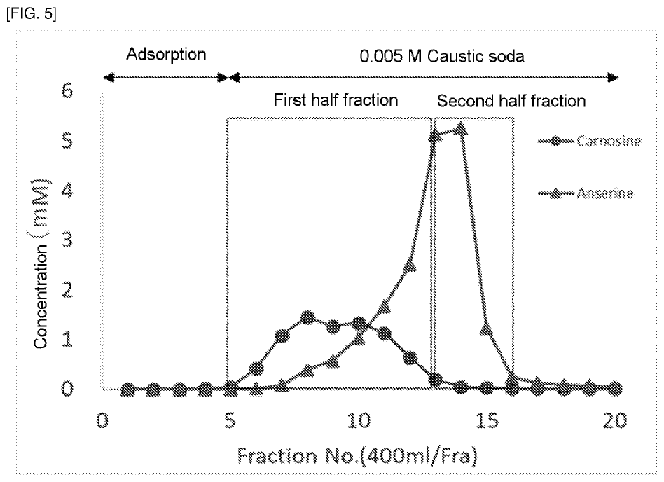

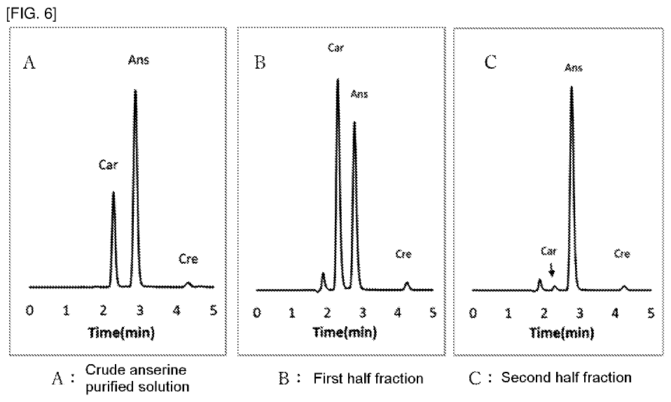

The method according to one embodiment of the present invention can, however, mutually separate each imidazole dipeptide in an ion exchange treatment solution of an animal extract regardless of the type of animal in the animal extract used. For example, when referring to FIG. 5, which shows the results in the case of subjecting the ion exchange treatment solution of the chicken extract to the method according to one specific embodiment of the present invention, the fraction numbers 5 to 7 can be used to obtain a purified imidazole dipeptide composition containing a highly pure carnosine and a very little anserine. To this contrary, the second half fraction can be used to obtain a purified imidazole dipeptide composition containing a highly pure anserine and a very little carnosine (see FIG. 6C).

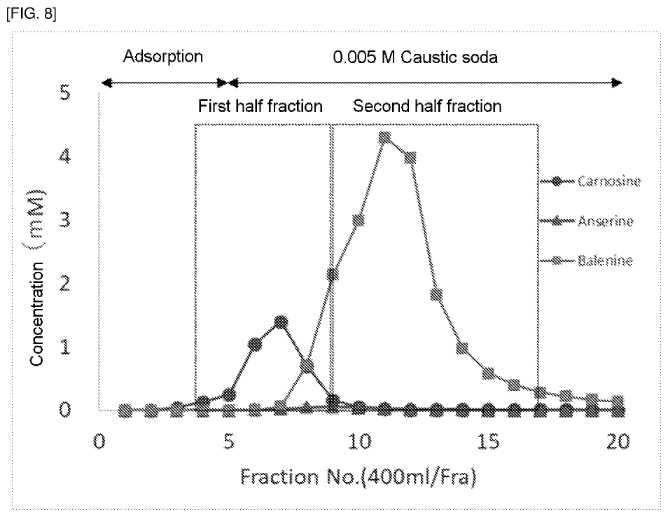

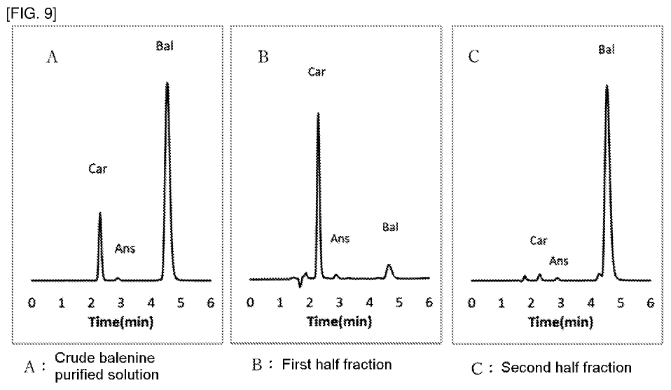

As the other example, when referring to FIG. 8, which shows the results in the case of subjecting the ion exchange treatment solution of the whale meat extract to the method according to one specific embodiment of the present invention, the first half fraction can be used to obtain a purified imidazole dipeptide composition containing a highly pure carnosine and a very little balenine (see FIG. 9B). To this contrary, the second half fraction can be used to obtain a purified imidazole dipeptide composition containing a highly pure balenine and a very little carnosine (see FIG. 9C).

As such, by applying the method according to one embodiment of the present invention, a purified imidazole dipeptide composition containing at high purity any one of imidazole dipeptides such as anserine, balenine and carnosine can be obtained from an animal extract treatment solution but the type of animals such as chicken and whale is not limited.

The method according to one embodiment of the present invention includes the following steps (1) and (2):

(1) subjecting an animal extract treatment solution containing at least two types of imidazole dipeptides to adsorption treatment carried out by bringing the solution into contact with a hydrophobic adsorption resin to adsorb the imidazole dipeptides onto the hydrophobic adsorption resin; and

(2) subjecting the hydrophobic adsorption resin adsorbing the imidazole dipeptides to elution treatment using an aqueous solution to mutually separate and collect imidazole dipeptides to purify an imidazole dipeptide.

The imidazole dipeptides are not particularly limited as long as they are those as normally known. For example, the imidazole dipeptide can be said to be a dipeptide in which a histidine or a histidine derivative having an imidazole group is bound to an amino acid. Specific examples of the imidazole dipeptide include anserine (β-alanyl-1-methylhistidine), carnosine (β-alanylhistidine), balenine (β-alanyl-3-methylhistidine) and homocarnosine (γ-aminobutyryl-L-histidine). Since the method according to one embodiment of the present invention employs a hydrophobic adsorption resin to perform adsorption treatment and elution treatment based on the difference in hydrophobicity between imidazole dipeptides, the imidazole dipeptides to be mutually separated are preferably imidazole dipeptides with the difference in hydrophobicity between them, more preferably carnosine and other imidazole dipeptides than carnosine, and still more preferably carnosine, and anserine or balenine.

The animal extract may be obtained by dissolving components contained in meat and other body parts of fish, birds, mammals and other animals in an extracting medium. The type of animal is not particularly limited as long as it is an animal that contains imidazole dipeptides in its meat or other body parts. Examples of the animal include bonito, tuna, salmon, eel, shark, cattle and chicken that contain anserine in large quantity; pig that contains carnosine in large quantity; and whale that contains balenine in large quantity. The animal extract is preferably derived from meat or muscles from livestock animals such as chicken, whale, cattle and pig, and fish such as salmon, bonito and tuna, since the meat and muscles contain a large content of imidazole dipeptides, and the animals are abundant in terms of resources, or are easy to breed.

The method of obtaining the animal extract is not particularly limited. The animal extract may be obtained by subjecting the animal parts containing imidazole dipeptides to known extraction methods such as water extraction, hot water extraction and supercritical extraction, or may be commercially available. The animal extract may be obtained by subjecting the resulting extract from the extraction method to processing treatment such as solid-liquid separation treatment, concentration treatment, drying treatment and dilution treatment to remove insoluble solids and impurities from the extract.

The animal extract is preferably subjected to demineralization treatment since in the subsequent ion exchange treatment, the yield loss may be reduced and the amount of imidazole dipeptides adsorbed per resin may be improved, resulting in a higher purity of imidazole dipeptides. For example, the demineralization treatment is preferably carried out using the electrodialysis demineralization device “DW-3E2 type” (manufactured by AGC Engineering) equipped with CMV-N/AMV-N as a cation exchange membrane/anion exchange membrane under the condition in which the target conductivity per 1 wt % of imidazole dipeptides is in the range between 2 mS/cm and 14 mS/cm, preferably about 5 mS/cm.

The animal extract treatment solution preferably contains at least two types of high purity imidazole dipeptides. The methods of obtaining the animal extract treatment solution from an animal extract include, but are not limited to, the method described in Patent Document 1, the method described in Patent Document 2 and the method described in the specification of Japanese patent application number JP 2019-235532 (the entire disclosure of each of which is incorporated by reference herein).

The content of imidazole dipeptides in the animal extract treatment solution is, as dry mass (solid content), preferably equal to or more than 70 wt %, and more preferably equal to or more than 80 wt %. Since the animal extract often contains creatinine, the content of creatinine in the animal extract treatment solution is, per mass of imidazole dipeptides, preferably equal to or less than 10 wt %, and more preferably equal to or less than 5 wt %.

The animal extract treatment solution in which the content of imidazole dipeptides is 80 wt % or more as dry mass (solid content) and the content of creatinine is 5 wt % or less per mass of imidazole dipeptides, which is one preferable embodiment of the animal extract treatment solution, can be obtained, for example, by the method described in the specification of JP 2019-235532. As such, one preferable embodiment of the animal extract treatment solution is the animal extract treatment solution obtained by subjecting an animal extract, preferably an animal extract subjected to demineralization treatment, to ion adsorption treatment using a strongly acidic cation exchange resin and elution treatment using an alkaline aqueous solution.

While the animal extract treatment solution contains at least two types of imidazole dipeptides in terms of mutual separation of each of imidazole dipeptides, the animal extract treatment solution may substantially contain any one type of imidazole dipeptides in terms of performing a higher degree of purification.

In order to ensure improved adsorption of the imidazole dipeptides on the hydrophobic adsorption resin during the adsorption step, the animal extract treatment solution has preferably a pH value of 7 to 10, and in view of the fact that the effective charge of the imidazole dipeptide is around zero, more preferably a pH value of 7.5 to 9.5 and still more preferably a pH value of 8.0 to 9.0. If the pH value of the animal extract treatment solution is out of the above range, the pH value may be adjusted using an acid or alkali.

[Step (1): Adsorption Treatment Step]

In the step (1), the animal extract treatment solution is subjected to adsorption treatment carried out by bringing the solution into contact with a hydrophobic adsorption resin to adsorb the imidazole dipeptides onto the hydrophobic adsorption resin.

The hydrophobic adsorption resin is not particularly limited as long as it is a synthetic resin having a hydrophobic porous structure but not having ion exchange groups. The hydrophobic adsorption resin is preferably an aromatic hydrophobic adsorption resin. The aromatic hydrophobic adsorption resin is a hydrophobic adsorption resin having a benzene ring as an adsorptive substituent. Examples of the aromatic hydrophobic adsorption resin include a hydrophobic adsorption resin with phenyl groups or phenylalkyl groups that may have substituents having the same or different structures as adsorptive substituents. The aromatic hydrophobic adsorption resin is preferably a hydrophobic adsorption resin having the structure represented in the following general formula (I).

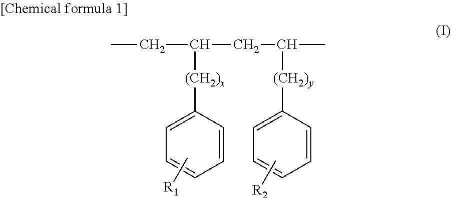

(wherein R₁ and R₂ are each independently hydrogen atom, halogen atom selected from the group consisting of F, Cl, Br and I, methyl group, ethyl group, methylene group or ethylene group, x is an integer of 0 to 2, and y is an integer of 0 to 2).

The hydrophobic adsorption resin may be produced by known methods or commercially available. Examples of the hydrophobic adsorption resin as being commercially available include “Sepabeads SP207”, “Sepabeads SP70”, “Sepabeads SP850”, “Sepabeads SP825L”, “Sepabeads SP700”, “DIAION HP20” and “DIAION HP21” (manufactured by Mitsubishi Chemical, respectively). Each of such aromatic hydrophobic adsorption resins can be preferably used in the method according to one embodiment of the present invention.

The method for bringing the animal extract treatment solution into contact with the hydrophobic adsorption resin is not particularly limited, as long as the imidazole dipeptides contained in the animal extract treatment solution can be adsorbed onto the hydrophobic adsorption resin. The method may be a batch method in which the hydrophobic adsorption resin is immersed in the animal extract treatment solution, or a column method in which the animal extract treatment solution is passed through a column packed with the hydrophobic adsorption resin. If the method is a simulated moving bed method, the collection rate of imidazole dipeptides may be enhanced.

Other adsorption conditions such as the content of the imidazole dipeptides contained in the animal extract treatment solution, the amount of the animal extract treatment solution loaded to the hydrophobic adsorption resin and the adsorption temperature may be set accordingly within the range of the adsorbent capacity of the hydrophobic adsorption resin employed since they can vary depending on the type and amount of the hydrophobic adsorption resin employed and other factors. In the case of passing the animal extract treatment solution through the column packed with the hydrophobic adsorption resin, the contact rate of the animal extract treatment solution through the hydrophobic adsorption resin is not particularly limited as long as the imidazole dipeptides contained in the animal extract treatment solution are adsorbed onto the hydrophobic adsorption resin. For example, the contact rate is preferably a flow rate in which SV is in the range between 0.5 and 10, preferably in the range between 1 and 5, at a temperature of 10° C. to 30° C., preferably at room temperature (about 20° C.).

For example, if 2 g of imidazole dipeptides are adsorbed onto 1 L of resin, the imidazole dipeptides may be adsorbed onto the hydrophobic adsorption resin by bringing the animal extract treatment solution having the amount of 1 RV to 10 RV, preferably 1 RV to 5 RV, into contact with the hydrophobic adsorption resin at a flow rate of SV 1 to SV 3 and at a temperature of 15° C. to 25° C., preferably at room temperature, wherein the content of imidazole peptides contained in the animal extract treatment solution is equal to or more than 0.01 wt %, preferably 0.05 wt to 1.0 wt %.

[Step (2): Elution Treatment Step]

In the step (2), the hydrophobic adsorption resin adsorbing the imidazole dipeptides is subjected to elution treatment using an aqueous solution. Through the step (2), it is possible to mutually separate and collect several types of imidazole dipeptides in the animal extract treatment solution, thereby obtaining a purified imidazole dipeptide composition containing a single high purity imidazole dipeptide.

The aqueous solution can be used as an eluent to mutually separate several types of imidazole dipeptides, depending on the hydrophobicity of each imidazole dipeptide. The aqueous solution may be an aqueous solution that is composed of 95 wt % or more of water and has a pH value of neutral to slightly alkaline. The aqueous solution is preferably selected from the group consisting of water, a dilute alkaline aqueous solution and a dilute organic solvent aqueous solution. The dilute alkaline aqueous solution may contain an alkaline substance and have a pH value of 8 to 12. The dilute organic solvent aqueous solution may be a mixture of a hydrophilic organic solvent and water.

While the type of alkaline substance contained in the dilute alkaline aqueous solution is not particularly limited, examples of the dilute alkaline aqueous solution include alkali metal salt hydroxide aqueous solutions such as a sodium hydroxide aqueous solution and a potassium hydroxide aqueous solution, and inorganic alkaline aqueous solutions such as an ammonia aqueous solution. From the viewpoint of the elution efficiency and collection rate of imidazole dipeptides, the alkaline aqueous solution is preferably an alkali metal salt hydroxide aqueous solution, more preferably a sodium hydroxide aqueous solution. For example, the dilute alkaline aqueous solution is preferably 0.001 M to 0.01 M alkali metal salt hydroxide aqueous solution, more preferably 0.001 M to 0.008 M alkali metal salt hydroxide aqueous solution, and still more preferably 0.002 M to 0.007 M alkali metal salt hydroxide aqueous solution.

While the hydrophilic organic solvent used for the dilute organic solvent aqueous solution are not particularly limited, examples of the organic solvent include lower aliphatic alcohols with 1 to 5 carbons such as methanol, ethanol, propyl alcohol and isopropyl alcohol; lower aliphatic ketones such as acetone and methylethyl ketone; and polyhydric alcohols with 2 to 5 carbons such as 1,3-butylene glycol, propylene glycol and glycerin. The organic solvent is preferably selected from the groups of consisting of methanol, ethanol, propyl alcohol, isopropyl alcohol and acetone. For example, the dilute organic solvent aqueous solution is preferably 0.01 wt % to 1.0 wt % of dilute organic solvent aqueous solution, more preferably 0.1 wt % to 0.8 wt % of dilute organic solvent aqueous solution, and still more preferably 0.2 wt % to 0.7 wt % of dilute organic solvent aqueous solution.

The amount of eluent used is not particularly limited. For example, when the animal extract treatment solution is passed through a column packed with 2 L of aromatic hydrophobic adsorption resin, several types of imidazole dipeptides adsorbed on the hydrophobic adsorption resin can be mutually separated in an efficient manner by passing 0.003 M to 0.006 M sodium hydroxide aqueous solution having the amount of 2 RV to 20 RV, preferably 5 RV to 10 RV, through the column at a flow rate of SV 1.0 to SV 3.0 and at a temperature of 15° C. to 25° C., preferably at room temperature.

The elution treatment may be carried out by stirring the hydrophobic adsorption resin packed and held in the column by an agitator or by blowing a gas into the column, or by gradually adding the eluent.

By carrying out the steps (1) and (2), it is possible to mutually separate and collect several types of imidazole dipeptides in the animal extract treatment solution, thereby obtaining a purified imidazole dipeptide composition containing a single high purity imidazole dipeptide. So long as the purified imidazole dipeptide composition is obtained by carrying out the steps (1) and (2), the purity of the imidazole dipeptide in the composition is not particularly limited. Examples of the purified imidazole dipeptide composition include a purified imidazole dipeptide composition in which the content of the specific single imidazole dipeptide is, relative to the whole content of imidazole dipeptides, 75 wt % or more, preferably 85 wt % or more, more preferably 90 wt % or more, and still more preferably 95 wt % or more.

In a case where the hot water extract of chicken breast is used as an animal extract, specific examples of the purified imidazole dipeptide composition include a purified imidazole dipeptide composition in which the content of anserine is, relative to the whole content of imidazole dipeptides (anserine, carnosine and balenine), 75 wt % or more, preferably 80 wt % or more, more preferably 90 wt % or more, still more preferably 95 wt % or more, and even still more preferably 97 wt % or more. In this case, in the purified imidazole dipeptide composition, the content of carnosine relative to the whole content of imidazole dipeptides may be 25 wt % or less, preferably 20 wt % or less, more preferably 10 wt % or less, and still more preferably 5 wt % or less. Furthermore, in this case, the yield of anserine would be able to become equal to or more than 50% based on the content of anserine in the animal extract treatment solution.

In another case where the hot water extract of whale meat is used as an animal extract, specific examples of the purified imidazole dipeptide composition include a purified imidazole dipeptide composition in which the content of balenine is, relative to the whole content of imidazole dipeptides (balenine, carnosine and anserine), 85 wt % or more, preferably 90 wt % or more, more preferably 95 wt % or more, and still more preferably 97 wt % or more. In this case, in the purified imidazole dipeptide composition, the content of carnosine relative to the whole content of imidazole dipeptides may be 25 wt % or less, preferably 20 wt % or less, more preferably 10 wt % or less, and still more preferably 5 wt % or less. Furthermore, in this case, the yield of balenine would be able to become equal to or more than 70% based on the content of balenine in the animal extract treatment solution.

The contents of anserine, balenine and carnosine are determined according to the method described in Examples below.

The purified imidazole dipeptide composition obtained through the steps (1) and (2) may be subjected to any treatments such as pH adjustment treatment, decolorization treatment, deodorization treatment, solid-liquid separation treatment, demineralization treatment, concentration treatment, aseptic treatment and dry treatment, for the use as a food material. Examples of such treatment include pH adjustment treatments in which the purified imidazole dipeptide composition obtained in the step (2) are adjusted to a pH value of 6 to 8, preferably around 7, with the use of acids such as hydrochloric acid; decolorization and/or deodorization treatments in which materials capable of adsorbing colored and/or odorous components, which include an activated carbon and a strongly basic ion exchange resin, are used; solid-liquid separation treatments such as filtration treatment using a ceramic filter; demineralization treatments using an electrodialysis membrane or a nanofiltration membrane; concentration treatments using an evaporator; aseptic treatments using a membrane filter; dry treatments using a spray dryer; and a combination of two or more of the above treatments to be subjected in turn. While each treatment is not particularly limited in terms of the conditions and procedures as long as the loss of imidazole dipeptides does not become more significant. Known methods for the treatment may be employed.

For example, the demineralization treatment of the purified imidazole dipeptide composition may be performed at a pH value of 8.0 or less using a nanofiltration membrane with a fractional molecular weight of 500 Da or less and/or a sodium chloride rejection rate (the rate at which sodium chloride is retained on the membrane) of 50% or less. Such a nanofiltration membrane is described in Table 3 of Patent Document 3. For example, when the purified imidazole dipeptide composition is subjected to the demineralization treatment, the salt concentration after the demineralization treatment is, as mass of sodium relative to mass of imidazole dipeptides, preferably less than or equal to 5 wt %, and more preferably less than or equal to 2 wt %.

The method according to one embodiment of the present invention may include various steps and operations before, after, or during the above steps as long as it can solve the problems of the present invention. The method according to one embodiment of the present invention preferably consists of, as the steps for purifying imidazole dipeptides, the step (1) of subjecting an animal extract treatment solution containing at least two types of imidazole dipeptides to adsorption treatment carried out by bringing the solution into contact with a hydrophobic adsorption resin to adsorb the imidazole dipeptides onto the hydrophobic adsorption resin; and the step (2) of subjecting the hydrophobic adsorption resin adsorbing the imidazole dipeptides to elution treatment using an aqueous solution to mutually separate and collect the imidazole dipeptides to purify an imidazole dipeptide. This means that in the method according to one embodiment of the present invention, no other steps are preferably included between the steps (1) and (2).

While a specific embodiment of the method of producing a purified imidazole dipeptide composition including the method of obtaining an animal extract treatment solution will be described below, the method according to the present invention is not limited thereto.

An acid is passed through a column packed with a strongly acidic cation exchange resin to convert the ion exchange group of the resin to the H type one, and then water is passed through the column, and further an aqueous alkali metal salt solution is passed through the column to convert the ion exchange group of the resin to the Na type one. Subsequently, the excess aqueous alkali metal salt solution is washed away by flowing water through the column.

Animal parts containing imidazole dipeptides are added to water, and the resulting mixture is subjected to hot water extraction treatment at a temperature in the range between 80° C. and 95° C. for tens of minutes to several hours. The obtained hot water extract is subjected as it stands or after subjected to demineralization treatment using an electrodialysis or nanofiltration membrane to concentration treatment and solid-liquid separation treatment, thereby obtaining an animal extract in which the content of imidazole dipeptides are in the range between 0.1 wt % and 1.0 wt %, the Brix value is in the range between 1.0% and 10.0%, and the pH value is in the range between 5.6 and 8.0.

The animal extract is flowed through the column packed with the Na type strongly acidic cation exchange resin with the amount of 1 RV to 10 RV at a flow rate of SV 1 to SV 3, and then water is flowed through the column with the amount of 0.5 RV to 5 RV to adsorb the imidazole dipeptides contained in the animal extract onto the strongly acidic cation exchange resin. The pH value in the column after this adsorption treatment is in the range between 5.6 and 8.0.

Subsequently, 0.1 N to 1.0 N alkali metal salt hydroxide aqueous solution is flowed through the column at a flow rate of SV 1 to SV 5 and with the amount of 1 RV to 5 RV to obtain high purity imidazole dipeptides as an eluate (an animal extract treatment solution). The pH value in the column after the elution treatment is in the range between 8.5 and 14.0.

The resulting animal extract treatment solution is adjusted to a pH value of 8 to 9 by adding an acid and then passed through a column packed with an aromatic hydrophobic adsorption resin with the amount of 1 RV to 10 RV at a flow rate of SV 1 to SV 5 at 10° C. to 30° C. to adsorb the imidazole dipeptides contained in the animal extract treatment solution onto the aromatic hydrophobic adsorption resin. The pH value in the column after this adsorption treatment is in the range between 8 and 9 in the same manner as that of the animal extract treatment solution used.

Subsequently, 0.001 M to 0.01 M alkali metal salt hydroxide aqueous solution as a dilute alkaline aqueous solution is flowed with the amount of 1 RV to 10 RV through the column at a flow rate of SV 1 to SV 5 at 10° C. to 30° C. to mutually separate several types of imidazole dipeptides. Each appropriate amount of fractions is collected and the imidazole dipeptide contained therein is then purified to obtain a purified imidazole dipeptide composition containing each high purity imidazole dipeptide. The pH value in the column after the elution treatment is in the range between 8 and 12 in the same manner as that of the dilute alkaline aqueous solution used.

The purified imidazole dipeptide composition may be sequentially subjected to pH adjustment treatment that adjusts the solution to near neutrality with the use of an acid, demineralization treatment using an electrodialysis membrane or nanofiltration membrane, concentration treatment using an evaporator, and sterile filtration treatment using a membrane filter with a pore size of 0.20 μm to 0.45 μm, to obtain a purified high purity imidazole dipeptide composition.

The dosage form of the purified imidazole dipeptide composition obtained by the method according to one embodiment of the present invention is not particularly limited and may be in either liquid form or solid form. In order to make the purified imidazole dipeptide composition suitable for a long-term storage, it is preferable to render the composition in the liquid form the composition in the powder form by subjecting the composition in the liquid form to drying treatment such as air-drying, decompression drying, freeze drying and spray drying.

The uses of the purified imidazole dipeptide composition obtained by the method according to one embodiment of the present invention are not particularly limited. The purified imidazole dipeptide composition has a large content of a certain imidazole dipeptide and a small content of the other imidazole dipeptides. For example, if the purified imidazole dipeptide composition contains high purity anserine, balenine or carnosine, the purified imidazole dipeptide composition may be used as a raw material for various compositions including oral compositions such as foods, drinks and pharmaceuticals, and topical compositions such as cosmetics, or as the composition itself, in anticipation of the physiological activities such as anti-fatigue effect, anti-oxidation effect, enhanced blood glucose level suppression effect, and cognitive function improvement effect achieved by the imidazole dipeptide contained.

While the content of the purified imidazole dipeptide composition in the food, drink and cosmetics is not particularly limited, examples of the content include an amount set in such a way that the amount of imidazole dipeptide becomes, as dry mass relative to the total amount of the food, drink or cosmetics, preferably 0.001 wt % or more, and more preferably from 0.1 wt % to 99 wt %.

The dosage form of the food and drink is not particularly limited, but includes, for example, the forms of liquid, powder, tablet, round, fine grain, granule, capsule, jelly, chewable and paste.

Specific examples of the food and drink may include, but not limited to, the followings: drinks, such as soft drinks, carbonated drinks, fruit drinks, vegetable juices, lactic acid bacteria drinks, milk drinks, soy milk, mineral water, tea drinks, coffee drinks, sports drinks, alcoholic drinks and jelly drinks; vegetable processed products such as tomato puree, canned mushrooms, dried vegetables and pickles; fruits processed products such as dried fruits, jams, fruit purees and canned fruits; spices such as curry powder, horseradish, ginger, spice blends and seasoning powders; noodles (including fresh and dried noodles) such as pasta, udon, soba noodles, ramen noodles, and macaroni; breads such as sliced breads, sweet breads, prepared breads and doughnuts; flour products such as alphalized rice, oatmeal, fu and batter flour; confectionery such as baked cakes, cookies, rice cakes, candies, chocolates, chewing gums, snack confectionery, chilled desserts, candied confectionery, Japanese cakes, western cakes, semi-baked cakes, pudding and ice cream; bean products such as azuki beans, tofu, natto, soybean flour, yuba (bean curd lees), cooked beans and peanuts; processed foods such as honey and royal jelly; meat products such as ham, sausage and bacon; dairy products such as yogurt, pudding, condensed milk, cheese, fermented milk, butter and ice cream; egg processed products; fish processed foods such as dried fish, kamaboko, chikuwa and fish sausage; processed seaweed such as dried seaweed, kelp and tsukudani; fish egg processed products such as cod roe, herring roe, salmon roe and karasumi; seasonings such as dashi broth, soy sauce, vinegar, mirin, consomme base, Chinese base, concentrated dashi, dressing, mayonnaise, ketchup and miso; edible fats and oils such as salad oil, sesame oil, linoleum oil and diacylglycerol and benibana oil; prepared foods such as soups (including powders and liquids), cooked food, retort food, chilled food and semi-cooked food (e.g., cooked rice stock, crab ball stock).

When the purified imidazole dipeptide composition according to one embodiment of the present invention is used to be blended into the cosmetics, the cosmetics may be used in the various forms such as lotion, emulsion, cream, gel and pack.

The present invention will now be described in further detail with reference to Examples, which are not intended to limit the present invention. The present invention may take various embodiments to the extent that the objectives of the present invention are achieved.

### Examples

### Example 1. Evaluation of Mutual Separation of Anserine and Carnosine

L-anserine (anserine purified product derived from salmon, manufactured by Tokai Bussan) and L-carnosine (manufactured by Hamari Chemicals) were used to prepare 100 ml of a mixture solution containing 200 μmol of each of them. The resulting mixture solution was passed through a column (diameter: 20 mm, height: 300 mm) packed with 50 ml of the aromatic synthetic adsorption resin “SEPABEADS SP207” (manufactured by Mitsubishi Chemical) at 20° C. and at SV 2 to adsorb anserine and carnosine onto the resin. Through the column, 0.005 M sodium hydroxide aqueous solution at 20° C. was passed with the amount of 6 RV and at SV 2 in order to collect fraction solutions every 10 ml to elute anserine and carnosine.

The concentrations of anserine and carnosine in the fraction solutions obtained were determined by HPLC. With HPLC, the column “InertSustain C18 (particle size: 5 μm, diameter: 4.6 mm, length: 150 mm)” (manufactured by GL Sciences) was employed, and an aqueous solution with 10 mM sodium phosphate (pH 6.5) was employed as the developing solvent. HPLC, “PU-2089” (manufactured by Nippon Spectroscope; flow rate: 1.0 ml/min, 25° C., injection volume: 5 μl, detector wavelength: 210 nm) was employed.

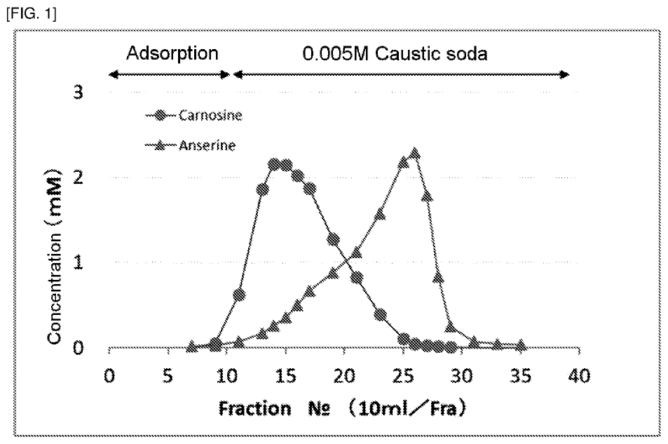

The example separation is shown in FIG. 1. As shown in FIG. 1, it was confirmed that carnosine was eluted first, and then anserine was eluted. Since each of carnosine and anserine was eluted with a different elution peak from each other, it was confirmed that the fraction solution with a high anserine content could be obtained by shifting the timing of collection and collecting the eluent after completion of the carnosine elution.

### Example 2. Evaluation of Eluent Used for Mutual Separation

The adsorption and elution of carnosine and anserine were confirmed in the same manner as in Example 1 by using the mixture solution containing anserine and carnosine as used in Example 1.

In this example, as the eluent, distilled water, 0.5 wt % ethanol aqueous solution, 0.01 M sodium hydroxide aqueous solution or 0.005 M sodium hydroxide aqueous solution was employed.

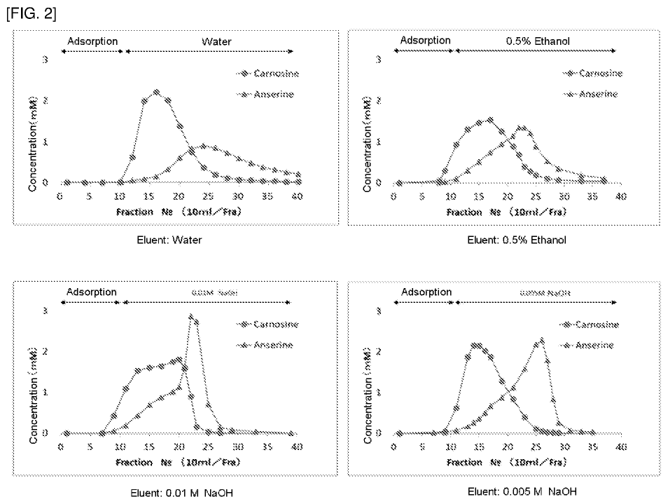

The example separation is shown in FIG. 2. As shown in FIG. 2, it was confirmed that each of carnosine and anserine was eluted with a different elution peak from each other when each of the above eluents was used.

### Example 3. Evaluation of Synthetic Adsorption Resin Used for Mutual Separation

L-anserine (anserine purified product derived from salmon, manufactured by Tokai Bussan) and L-carnosine (manufactured by Hamari Chemicals) were used to prepare 100 ml of a mixture solution containing 50 μmol (0.5 mM) of each of them. The resulting mixture solution was passed through a column (diameter: 20 mm, height: 300 mm) packed with 50 ml of the aromatic synthetic adsorption resin “DIAION HP20” (manufactured by Mitsubishi Chemical) at 20° C. and at SV 2 to adsorb anserine and carnosine onto the resin. Through the column, distilled water with the amount of 3 RV at 20° C. was passed at SV 2 in order to collect fraction solutions every 10 ml to elute anserine and carnosine.

The concentrations of anserine and carnosine in the fraction solutions obtained were determined by HPLC in the same manner as in Example 1.

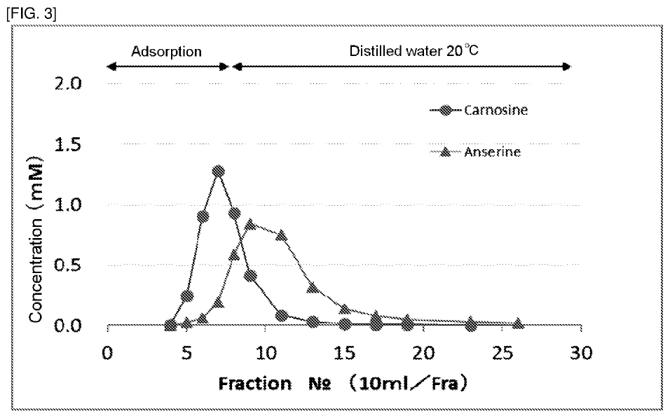

The example separation is shown in FIG. 3. As shown in FIG. 3, it was found that each of the aromatic synthetic adsorption resins used could be used to mutually separate carnosine and anserine.

### Example 4. Evaluation of Mutual Separation of Carnosine and Anserine Using Chicken Meat

Chicken breast meat was subjected to hot water extraction to obtain a chicken extract. The resulting chicken extract was subjected to diatomaceous earth filtration treatment and then diluted with water to obtain a crude chicken extract in which the Brix value was 7.5%, the content of anserine was 0.53 wt %, and the content of carnosine was 0.22 wt %. The obtained crude chicken extract was passed through a column packed with 1,000 ml of the strongly acidic cation exchange resin “DIAION SK1B” (manufactured by Mitsubishi Chemical), which was converted to the form of Na type using 10% NaCl in advance, with the amount of 4 RV and at SV 2 to adsorb the imidazole dipeptides onto the resin. After the adsorption treatment, RO water with the amount of 1 RV was passed through the column at SV 2, and 0.4 M sodium hydroxide aqueous solution was passed through the column with the amount of 2 RV and at SV 2 to elute the imidazole dipeptides adsorbed on the resin to obtain a crude anserine purified solution (ion exchange treatment solution) derived from chicken. For reference, using the separately prepared chicken extract and ion exchange treatment solution, and the filtration solution (ion exchange treatment+NF membrane treatment solution) that was obtained by subjecting the ion exchange treatment solution to nanofiltration treatment with a nanofiltration membrane having a fractional molecular weight of 500 Da or less and a sodium chloride rejection rate of 50% or less, HPLC chromatograms were measured in the same manner as in Example 1. The results are shown in FIG. 4 and Table 1.

TABLE 1

| | Concentration per solid content | | | Carnosine/ |
|---|---|---|---|---|
| Purification | (%) | | | Anserine |
| method | Anserine | Carnosine | Creatinine | (%) |
| Chicken extract | 8 | 3 | 3 | 36 |
| Ion exchange | 51 | 16 | 3 | 31 |
| treatment solution | | | | |
| Ion exchange | 63 | 18 | 2 | 28 |
| treatment + NF | | | | |
| membrane | | | | |
| treatment solution | | | | |

The crude anserine purified solution from chicken (anserine: 2,002 mg, carnosine: 757 mg) was passed through a column (diameter: 550 mm, height: 1,000 mm) packed with 1,000 ml of the aromatic synthetic adsorption resin “SEPABEADS SP207” (manufactured by Mitsubishi Chemical) with the amount of 2 RV and at SV 2 to carry out adsorption treatment.

Through the column, 0.005 M sodium hydroxide aqueous solution was then passed with the amount of 6 RV and at SV 2 to carry out elution treatment. The fraction solutions were collected every 400 ml from the eluent, and the concentrations of anserine and carnosine in the fraction solutions obtained were determined by HPLC in the same manner as in Example 1. The example separation is shown in FIG. 5.

As shown in FIG. 5, it was confirmed that carnosine was eluted first, and then anserine was eluted. It was assumed that since each of carnosine and anserine was eluted with an elution peak mutually separated from the other one, the fraction solution with a high anserine ratio could be obtained by collecting anserine after completion of the carnosine elution.

The eluate was divided into two fractions before and after the point at which the mass ratio of carnosine/anserine fell below 10%, and the two fractions were collected as a first half fraction and a second half fraction, respectively. The HPLC chromatograms of the fractions are shown in FIG. 6. The collection rate and composition ratio of anserine, carnosine and balenine are shown in Table 2.

TABLE 2

| | | Ans | | Car | | Bal | | Ratio (by weight) % | | |
|---|---|---|---|---|---|---|---|---|---|---|
| | Fraction | mg | Yield % | mg | Yield % | mg | Yield % | Ans | Car | Bal |
| Loaded | Crude anserine | 2001 | — | 757 | — | 0 | — | 72.6 | 27.4 | 0.0 |
| | purified solution | | | | | | | | | |
| Collected | First half fraction | 582 | 29 | 654 | 86 | 0 | — | 47.1 | 52.9 | 0.0 |
| Collected | Second half fraction | 1123 | 56 | 34 | 4 | 0 | — | 97.1 | 2.9 | 0.0 |

As shown in FIG. 6 and Table 2, the collection rate of anserine relative to the loading amount was 29 wt % in the first half fraction while the collection rate was 56 wt % in the second half fraction. The composition ratio of imidazole dipeptides was, by % by weight, anserine:carnosine=47.1:52.9 for the first half fraction and anserine: carnosine=97.1:2.9 for the second half fraction.

Therefore, as the second half fraction, it was able to obtain a composition with a high anserine content in which the content of anserine was equal to or more than 90 wt % among imidazole dipeptides.

### Example 5. Evaluation of Mutual Separation of Carnosine and Balenine Using Whale Meat

To 1,500 g of minke whale breast meat (derived from Iceland), 3,000 g of city water was added, and the resulting mixture was subjected to hot extraction treatment at 90° C. for 60 minutes to obtain a whale meat extract. The resulting whale meat extract was subjected to diatomaceous earth filtration treatment to obtain a crude whale extract of 2,800 g in which the Brix value was 1.7%, the content of balenine was 0.48 wt %, and the content of carnosine was 0.09 wt %. The crude whale extract was subjected to ion exchange treatment in the same manner as in Example 4 to obtain a crude balenine purified solution derived from whale (ion exchange treatment solution). For reference, using the separately prepared whale meat extract and ion exchange treatment solution, and the filtration solution (ion exchange treatment+NF membrane treatment solution) that was obtained by subjecting the ion exchange treatment solution to nanofiltration treatment with the same nanofiltration membrane as in Example 4, HPLC chromatograms were measured in the same manner as in Example 1. The results are shown in FIG. 7 and Table 3.

TABLE 3

| | Concentration per solid content | | Carnosine/ |
|---|---|---|---|
| Purification | (%) | | Balenine |
| method | Balenine | Carnosine | (%) |
| Whale meat extract | 26 | 3 | 12 |
| Ion exchange | 59 | 9 | 15 |
| treatment solution | | | |
| Ion exchange | 68 | 11 | 16 |
| treatment + NF | | | |
| membrane | | | |
| treatment solution | | | |

The crude balenine purified solution from whale (balenine: 2,001 mg, carnosine: 359 mg, anserine: 19 mg) was used to carry out adsorption treatment and elution treatment in the same manner as in Example 4. The fraction solutions were collected every 400 ml from the eluent, and the concentrations of balenine, carnosine and anserine in the fraction solutions obtained were determined by HPLC in the same manner as in Example 1. The example separation is shown in FIG. 8.

As shown in FIG. 8, it was confirmed that carnosine was eluted first, and then balenine was eluted. It was assumed that since each of carnosine and balenine was eluted with an elution peak mutually separated from the other one, the fraction solution with a high balenine ratio could be obtained by collecting balenine after completion of the carnosine elution.

The eluate was divided into two fractions before and after the point at which the mass ratio of carnosine/balenine fell below 10%, and the two fractions were collected as a first half fraction and a second half fraction, respectively. The HPLC chromatograms of the fractions are shown in FIG. 9. The collection rate and composition ratio of anserine, carnosine and balenine are shown in Table 4.

TABLE 4

| | | Ans | | Car | | Bal | | Ratio (by weight) % | | |
|---|---|---|---|---|---|---|---|---|---|---|
| | Fraction | mg | Yield % | mg | Yield % | mg | Yield % | Ans | Car | Bal |
| Loaded | Crude balenine | 18 | — | 359 | — | 2002 | — | 0.8 | 15.1 | 84.2 |
| | purified solution | | | | | | | | | |
| Collected | First half fraction | 9 | 50 | 316 | 88 | 76 | 4 | 2.2 | 78.8 | 19.0 |
| Collected | Second half fraction | 14 | 78 | 25 | 7 | 1630 | 81 | 0.8 | 1.5 | 97.7 |

As shown in FIG. 9 and Table 4, the collection rate of balenine relative to the loading amount was 4 wt % in the first half fraction while the collection rate was 81 wt % in the second half fraction. The composition ratio of imidazole dipeptides was, by % by weight, balenine:carnosine:anserine=19.0:78.8:2.2 for the first half fraction and balenine:carnosine:anserine=97.7:1.5:0.8 for the second half fraction.

Therefore, as the second half fraction, it was able to obtain a composition with a high balenine content in which the content of balenine was equal to or more than 90 wt % among imidazole dipeptides.

### Example 6. Evaluation of Mutual Separation of Carnosine, Anserine and Balenine Using Pork

To 2,000 g of pork thigh (derived from Japan), 2,000 g of city water was added, and the resulting mixture was subjected to hot extraction treatment at 90° C. for 60 minutes to obtain a pork extract. The resulting pork extract all was subjected to diatomaceous earth filtration treatment to obtain a crude pork extract of 2,450 g in which the Brix value was 1.9%, the content of carnosine was 0.16 wt %, the content of anserine was 0.01 wt %, and the content of balenine was 0.01 wt %. The crude pork extract was subjected to ion exchange treatment in the same manner as in Example 4 to obtain a crude carnosine purification solution from pork (ion exchange treatment solution).

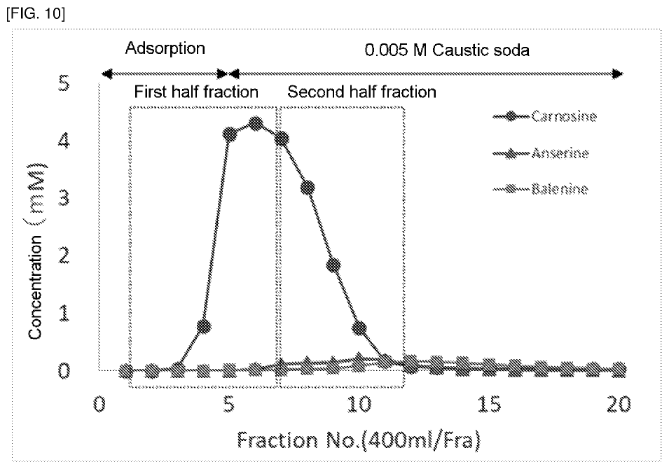

The crude carnosine purified solution from pork (carnosine: 1,786 mg, anserine: 116 mg, balenine: 132 mg) was used to carry out adsorption treatment and elution treatment in the same manner as in Example 4. The fraction solutions were collected every 400 ml from the eluent, and the concentrations of carnosine, anserine and balenine in the fraction solutions obtained were determined by HPLC in the same manner with in Example 1. The example separation is shown in FIG. 10.

As shown in FIG. 10, it was confirmed that carnosine was eluted first, and then anserine and balenine were eluted. It was assumed that since each of them was eluted with an elution peak mutually separated from the other ones, the fraction solution with a high carnosine ratio could be obtained by collecting carnosine before anserine and balenine were eluted.

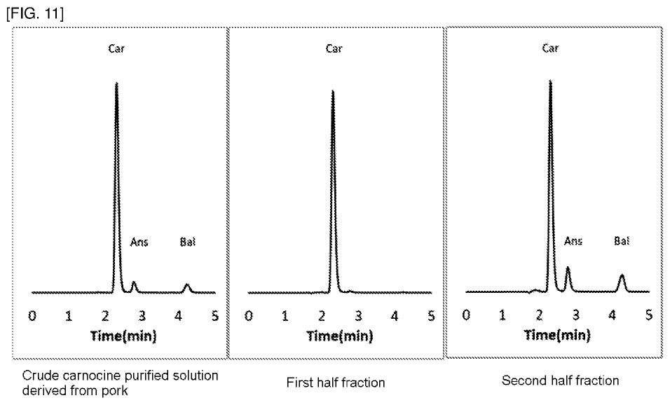

The eluate was divided into two fractions before and after the point at which the mass ratio of (anserine+balenine)/carnosine rose beyond 1%, and the two fractions were collected as a first half fraction and a second half fraction, respectively. The HPLC chromatograms of the fractions are shown in FIG. 11. The collection rate and composition ratio of carnosine, anserine or balenine are shown in Table 5.

TABLE 5

| | | Ans | | Car | | Bal | | Ratio (by weight) % | | |
|---|---|---|---|---|---|---|---|---|---|---|
| | Fraction | mg | Yield % | mg | Yield % | mg | Yield % | Ans | Car | Bal |
| Loaded | Crude Carnosine | 116 | — | 1786 | — | 132 | — | 5.7 | 87.8 | 6.5 |
| | purified solution | | | | | | | | | |
| Collected | First half fraction | 0 | 0 | 1223 | 68 | 0 | 0 | 0.0 | 100.0 | 0.0 |
| Collected | Second half fraction | 88 | 76 | 570 | 32 | 92 | 70 | 11.7 | 76.0 | 12.3 |

As shown in FIG. 11 and Table 5, the collection rate of carnosine relative to the loading amount was 68 wt % in the first half fraction while the collection rate was 32 wt % in the second half fraction. The composition ratio of imidazole dipeptides was, by % by weight, carnosine:anserine:balenine=100:0:0 for the first half fraction and carnosine:anserine:balenine=76.0:11.7:12.3 for the second half fraction.

Therefore, in the first half fraction, it was able to obtain a composition with a high carnosine content in which the content of carnosine was equal to or more than 95 wt % among imidazole dipeptides.

### INDUSTRIAL APPLICABILITY

The present invention is useful in the fields of foods and beverages, pharmaceuticals, cosmetics, quasi-pharmaceutical products and the like, and particularly has advantages in being capable of producing anti-fatigue compositions, anti-oxidation compositions, enhanced blood glucose level suppression compositions, cognitive function improvement compositions or being used for raw materials for these compositions.

### CROSS-REFERENCE OF RELATED APPLICATIONS

The present application claims the benefit of priority to Japanese Patent Application No. 2020-080065, filed on Apr. 30, 2020, the disclosure of which is incorporated herein by reference in its entirety.

1. A method of producing a purified imidazole dipeptide composition, comprising the steps of:
(1) subjecting an animal extract treatment solution comprising at least two types of imidazole dipeptides to adsorption treatment carried out by bringing the solution into contact with a hydrophobic adsorption resin to adsorb the imidazole dipeptides onto the hydrophobic adsorption resin; and
(2) subjecting the hydrophobic adsorption resin adsorbing the imidazole dipeptides to elution treatment using an aqueous solution to mutually separate and collect the imidazole dipeptides to purify an imidazole dipeptide.

2. The method according to claim 1, wherein the at least two types of imidazole dipeptides comprise carnosine and an imidazole dipeptide different from carnosine.

3. The method according to claim 1, wherein the at least two types of imidazole dipeptides comprise carnosine, and anserine or balenine.

4. The method according to claim 1, wherein the hydrophobic adsorption resin is an aromatic hydrophobic adsorption resin.

5. The method according to claim 1, wherein the aqueous solution is at least one aqueous solution selected from the group consisting of water, a dilute alkaline aqueous solution and a dilute organic solvent aqueous solution.

6. The method according to claim 5, wherein the dilute alkaline aqueous solution is 0.001 M to 0.008 M sodium hydroxide aqueous solution, and the dilute organic solvent aqueous solution is 0.1 wt % to 0.8 wt % ethanol aqueous solution.

7. The method according to claim 1, wherein the animal extract treatment solution is obtained by subjecting an animal extract to ion adsorption treatment using a strongly acidic cation exchange resin followed by elution treatment using an alkaline aqueous solution.

8. The method according to claim 7, wherein the animal extract is an animal extract subjected to demineralization treatment.

9. The method according to claim 7, wherein the animal extract is derived from meat of at least one animal selected from the group consisting of chicken, whale, cattle, pig, salmon, bonito and tuna.

10. The method according to claim 8, wherein the animal extract is derived from meat of at least one animal selected from the group consisting of chicken, whale, cattle, pig, salmon, bonito and tuna.
